(19) United States
(12) Patent Application Publication
(10) Pub. No.: US 2022/0259272 A1
(43) Pub. Date: Aug. 18, 2022

(54) PHOTOACTIVE COMPOSITIONS FOR THERAPUETIC AGENT DELIVERY

(71) Applicant: The University of North Carolina at Chapel Hill, Chapel Hill, NC (US)

(72) Inventors: David LAWRENCE, Chapel Hill, NC (US); Brianna VICKERMAN, Chapel Hill, NC (US)

(21) Appl. No.: 17/618,944

(22) PCT Filed: Jun. 12, 2020
(86) PCT No.: PCT/US2020/037480

§ 371 Date: Dec. 14, 2021

Related U.S. Application Data
(60) Provisional application No. 62/861,799, filed on Jun. 14, 2019.

(51) Int. Cl. C07K 14/435; A61K 47/65; A61K 47/69; A61K 41/00

(52) U.S. Cl. CPC C07K 14/43572; A61K 41/0028; A61K 47/6901; A61K 47/65

(57) ABSTRACT

In one aspect, photoactive compositions are described herein for targeted or selective delivery of therapeutic agents to diseased tissue and/or diseased sites within a patient. A photoactive composition comprises a membrane-based carrier and a photoactivated lytic component coupled to the membrane-based carrier, the photoactivated lytic component comprising a lysing agent and photolabile lytic blocking agent.

### RELATED APPLICATION DATA

The present application claims priority pursuant to Article 8 of the Patent Cooperation Treaty to U.S. Provisional Patent Application Ser. No. 62/861,799 filed Jun. 14, 2019, which is incorporated herein by reference in its entirety.

### FIELD

The present invention relates to compositions and methods for the delivery of therapeutic agents to a diseased site in a patient and, in particular, to photoactive compositions for targeted delivery to the diseased site.

### BACKGROUND

Various compositions have been employed for the delivery of therapeutic agents to diseased tissue and/or diseased sites in a patient. Unfortunately, a number of such compositions lack specificity for the diseased site and/or exhibit short circulatory lifetimes.

Myocardial infarction, ischemic stroke, peripheral arterial occlusion, and pulmonary embolism due to blood clots (thrombi), for example, are treated with tissue plasminogen activator (tPA) in a small fraction of patients. tPA is an enzyme that activates an endogenous protein (plasminogen) to generate plasmin which, in turn dissolves the clot. Notably, massive amounts of tPA (1 mg/kg, up to 100 mg) are required to have the desired effect due to the short circulatory lifetime (5 min) and systemic distribution of the enzyme. The latter is responsible for serious side effects, most notably catastrophic intracerebral hemorrhage (˜10% of patients). Furthermore, even at these high doses, refractory clots are a common problem. There is intense interest in identifying solutions to short circulatory lifespan and off-target activity of tPA and other therapeutic agents for treating diseased tissue, including chemotherapeutic agents.

### SUMMARY

In view of these disadvantages, photoactive compositions are described herein for targeted or selective delivery of therapeutic agents to diseased tissue and/or diseased sites within a patient. A photoactive composition comprises a membrane-based carrier and a photoactivated lytic component coupled to the membrane-based carrier, the photoactivated lytic component comprising a lysing agent and photolabile lytic blocking agent. As described further herein, a photolabile lytic blocking agent inhibits or precludes the activity of the lysing agent. Accordingly, when in the presence of the photolabile lytic blocking agent, the lysing agent does not lyse the membrane-based carrier. However, exposure of the blocking agent to light of the proper wavelength induces loss of the lytic blocking character, thereby permitting the lysing agent to lyse the membrane-based carrier. Photolabile lytic blocking agents, for example, can absorb visible and/or infrared radiation. In some embodiments, a photolabile lytic blocking agent can absorb radiation having wavelength of 600-1200 nm or 750-1000 nm.

Lysing of the membrane-based carrier releases the contents of the carrier. The membrane-based carrier, for example, can comprise one or more therapeutic agents. Therapeutic agents can comprise biomolecules, including proteins, polysaccharides, peptides and/or nucleic acids. Therapeutic agents can also comprise various pharmaceutical compositions, including small molecule drugs, such as cytotoxins and chemotherapeutics. Release of therapeutic agents upon light induced activation of the lysing agent can permit targeted and controlled delivery of such agents to diseased tissue and/or diseased sites in a patient. In some embodiments, therapeutic agents released by photoactive composition described herein can treat a variety of conditions including, but not limited to, blood clots, cancerous tissue, and/or inflamed tissue. Table I provides some non-limiting embodiments of therapeutic agents carried by photoactive compositions described herein.

TABLE I
Therapeutic Agents

Tissue plasminogen activator (tPA)
Urokinase
Tumor necrosis factor-Related Apoptosis-Inducing Ligand (TRAIL)
Vascular Endothelial Growth Factor (VEGF)
truncated Tissue Factor (tTA)
Saporin
Tissue Necrosis Factor (TNF-α)
Hyaluronidase
Bradykinin
α-Melanocyte Stimulating Hormone
Angiotension-(1-7)
Qk - (mimic of VEGF)
PAR agonists
Auristatins

In some embodiments, the lysing agent and photolabile lytic blocking agent are coupled to the membrane of the carrier. The lysing agent and photolabile lytic blocking agent can be independently coupled to the membrane, in some embodiments. Alternatively, the lysing agent and photolabile blocking agent are coupled together constituting a single species. In such embodiments, the single species comprising the lysing agent and photolabile blocking agent can be coupled to the membrane of the carrier.

A photolabile lytic blocking agent can comprise one or more light-cleavable moieties blocking activity of the lysing agent (photolytic moiety). Specific chemical identity of a photolabile lytic blocking agent can be dependent on various considerations, including the chemical identity of the lysing agent and chemical identity of the membrane-based carrier. In some embodiments, a photolytic blocking agent comprises a transition metal complex. A photolytic blocking agent, for example, can comprise a cobalamin derivative. The transition metal complex can comprise one or more light-cleavable ligands blocking the activity of lysing agent. Light-cleavable moieties can have any composition and structure for blocking activity of the lysing agent. In some embodiments, the light-cleavable moiety can comprise a biomolecular species, such as a peptide or protein for blocking activity of the lysing agent. In some embodiments, the light-cleavable moieties can participate in one or more interactions with the lysing agent including, but not limited to, van der Waals interactions, hydrogen bonding and/or electrostatic interactions. Additionally, in some embodiments, a light cleavable-moiety is covalently bonded to the lysing agent.

The lysing agent can comprise any species consistent with the objective of the present invention. Specific chemical identity of the lysing agent can be dependent on several considerations, including identity of the membrane based carrier and identity photolabile lytic blocking agent. In some embodiments, the lysing agent can comprise one or more biomolecular species, such as a protein or peptide. The lysing agent, for example, can comprise a VA2 membrane active peptide, including melittin or a derivative thereof. A derivative of melittin, in some embodiments, can include one or more amino acid substitutions and/or modifications to an amino acid side chain. Other cationic peptides may serve as lysing agents. Additionally, non-biomolecular species may serve as lysing agents, in some embodiments.

Moreover, the membrane-based carrier can be a lipid membrane-based carrier, in some embodiments. Lipid membrane-based carriers can comprise cells, liposomes, exosomes, and/or related lipid bilayer entities.

In another aspect, a therapeutic composition comprises a first photoactive composition including a membrane-based carrier containing a first therapeutic agent, and a photoactivated lytic component coupled to the membrane-based carrier, the photoactivated lytic component comprising a lysing agent and photolabile lytic blocking agent. The therapeutic composition also comprises a second photoactive composition including a membrane-based carrier containing a second therapeutic agent, and a photoactivated lytic component coupled to the membrane-based carrier, the photoactivated lytic component comprising a lysing agent and photolabile lytic blocking agent, wherein the photolabile blocking agents of the first and second photoactive compositions have differing absorption spectra. In some embodiments, the photolabile lytic blocking agents can be modified with dyes or other spectral species to alter the absorption spectra.

Differing absorption spectra between the photolabile lytic blocking agents of the first and second compositions can permit tailoring of the temporal release characteristics of the first and second membrane-based carriers. The first and second photoactive compositions, for example, can be irradiated for sequential lysing and release of the therapeutic agents contained therein. In other embodiments, the first and second photoactive compositions can be irradiated for simultaneous lysing and therapeutic agent release. The ability to tailor temporal release characteristics of the first and second membrane-based carriers can enable proper timing for the release of synergistic therapeutic agents for effective treatment of diseased tissues or diseased sites in a patient. Any number of photoactive compositions employing photolabile lytic blocking agents of differing absorption spectra are contemplated herein.

In another aspect, methods of treatment are described herein. Such methods comprise delivering a photoactive composition to a diseased site, the photoactive composition comprising a membrane-based carrier containing one or more therapeutic agents, and a photoactivated lytic component coupled to the membrane-based carrier, the photoactivated lytic component comprising a lysing agent and photolabile lytic blocking agent. The lysing agent is activated via irradiating the photolabile lytic blocking agent, and the membrane-based carrier is lysed to release the one or more therapeutic agents.

These and other embodiments are further described in the following detailed description and attached Appendix.

### BRIEF DESCRIPTION OF THE DRAWINGS

FIG. 1 illustrates light activation of photoactive compositions described herein for the targeted release of therapeutic agents, according to some embodiments.

FIG. 2A illustrates red blood cell lysis according to various architectures.

FIG. 2B illustrates spatially resolved photo-hemolysis of red blood cell via photoactive compositions described herein, according to some embodiments.

FIG. 2C provides an assessment of photo-hemolysis of photoactive compositions described herein as a function of laser power, according to some embodiments.

FIG. 2D illustrates response of photoactive compositions described herein to radiation relative to control compositions, the photoactive compositions comprising liposome carriers.

FIG. 3A illustrates response of photoactive compositions described herein to radiation, according to some embodiments.

FIG. 3B provides results of light activation of photoactive compositions described herein for the targeted release of thrombin, according to some embodiments.

FIG. 3C provided his provides results of light activation of photoactive compositions described herein for the targeted in vivo release of thrombin, according to some embodiments.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Example 1—Photoactive Composition

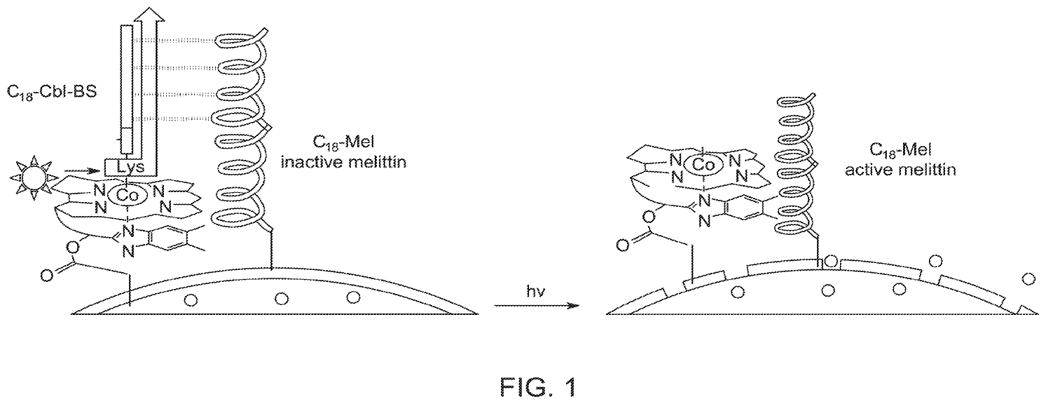

In the present example, red blood cells (RBCs) were engineered to convey and subsequently release internal protein cargo in response to pre-assigned wavelengths of light. The design strategy employs a photoactivatable, membrane-embedded, hemolytic peptide as illustrated in FIG. 1. As illustrated in FIG. 1 an assembly of a photo-hemolysis trigger is coupled to the surface of RBCs. Both human and mouse RBCs were first internally loaded with a therapeutic protein (circles) by sequential treatment with hypotonic conditions (pore formation), the protein of interest, and isotonic buffer (pore resealing). Simultaneous exposure of RBCs to C₁₈-Cbl-BS and C₁₈-Mel generates a photoresponsive RBC construct (FIG. 1, left). Upon illumination (525 nm), the blocking segment (BS) is released from the Cbl, generating active melittin (Mel), subsequent hemolysis, and release of the internal protein therapeutic. C₁₈-Cbl-Cy5BS is an analog of C₁₈-Cbl-BS that responds to 660 nm exposure.

Melittin (Mel), a 26-residue alpha-helical peptide, is a potent hemolytic agent that is a key component of European honey bee (Apis mellifera) venom. In the present example, the pronounced affinity of melittin for RBCs and its subsequent hemolytic activity were exploited by transforming melittin into a light-sensitive hemolytic agent.

Cobalamin (Cbl), also known as vitamin B12, is primarily available as one of four derivatives, each of which contains a different substituent on the Co metal of the corrin ring. The Co—C bond of methyl and adenosyl Cbl derivatives undergo homolytic cleavage upon illumination in the 330-550 nm range. In view of this photolabile chemistry, a conceptually distinct strategy was employed to prepare a melittin-based photo-hemolytic trigger. Mel is biosynthesized in honeybees as promelittin, a non-hemolytic peptide comprised of the positively charged Mel and a negatively charged inhibitory prodomain [Blocking Segment (BS)]. Mel and BS were synthesized as separate entities to create the photolytic trigger. The trigger was comprised of two entities: (1) Mel acylated at its N-terminus with stearic acid (C₁₈). The stearyl lipid moiety of C₁₈-Mel (FIG. 1) was employed to anchor Mel to the surface of RBCs. (2) The BS is covalently appended to a butyric acid linker, which in turn is attached to the Co of a C₁₈-modified Cbl (C₁₈-Cbl-BS; FIG. 1). RBCs were exposed to a premixed combination of C₁₈-Mel and C₁₈-Cbl-BS, which ensured that the C₁₈-Mel was not hemolytically active (FIG. 1). Upon illumination, the Co—C of C₁₈-Cbl-BS was designed to undergo photolysis, releasing the BS peptide from the RBC surface and thereby activating Mel, which promoted RBC lysis and the liberation of internally loaded protein contents. Protein therapeutics were readily introduced into RBCs under low salt conditions, which generates pores in the erythrocyte plasma membranes. Restoration of isotonicity reseals the pores and entraps the protein drug.

### Example 2—Photoactivated Release of Protein

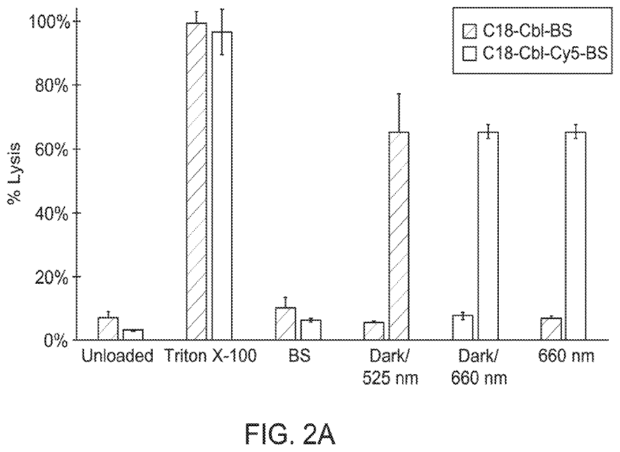

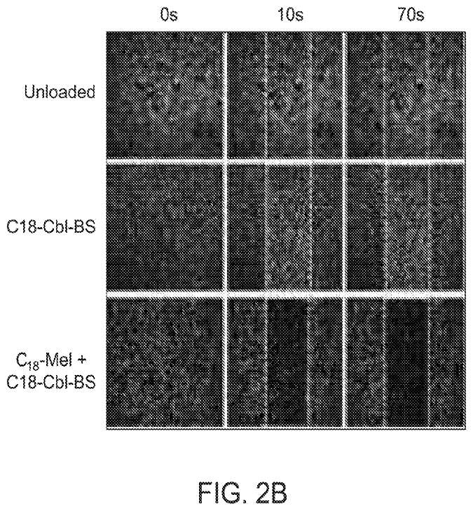

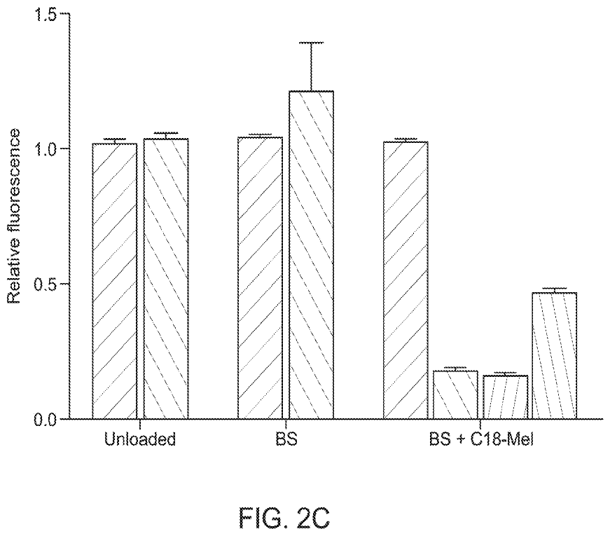

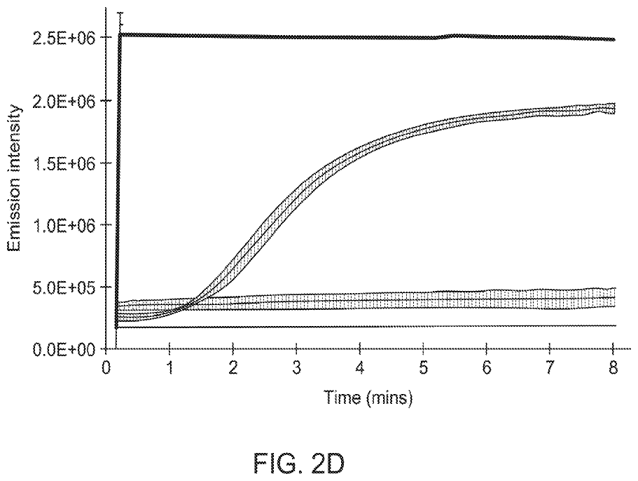

The release of bovine serum albumin-Texas Red (BSA-TxRed, FIG. 2A) from human RBCs was initially demonstrated. Light-triggered BSA-TxRed/RBC hemolysis was quantified via fluorescence of the supernatant. The results are compared relative to RBCs that lack the surface-anchored photolytic trigger (unloaded) and RBCs that were completely lysed by detergent (Triton X-100). These controls set the minimum and maximum supernatant fluorescence, respectively. Minimal fluorescence (which was taken as minimal lysis) was observed when RBC surface contains only C₁₈-Cbl-BS or C₁₈-Cbl-Cy5BS. These results demonstrated that C₁₈-Mel is essential for hemolysis. In addition, RBCs containing both C₁₈-Cbl-BS (or C₁₈-Cbl-Cy5BS) and C₁₈-Mel without light exposure (Dark) likewise displayed minimal lysis, thereby demonstrating a dependence on light for hemolysis. Finally, co-loading C₁₈-Cbl-BS and C₁₈-Mel onto RBCs and subsequent exposure to 525 nm light resulted in hemolysis that is 66±12% of that observed with Triton X-100.

A key challenge in utilizing photosensitive therapeutics is developing compounds that are responsive to wavelengths that can effectively penetrate tissue (600-900 nm), known as the optical window of tissue. That photolytic wavelength can tuned to the optical window of tissue by appending long-wavelength fluorophores to Cbl. Long wavelength photolysis was investigated by using a Cy5 derivatized BS peptide (C₁₈-Cbl-Cy5BS) that absorbs light at 660 nm. It was noted that, in the absence of the Cy5 fluorophore, RBCs containing C₁₈-Mel and C₁₈-Cbl-BS do not suffer hemolysis upon exposure to 660 nm (FIG. 2A). By contrast, RBCs loaded with the Cy5 antenna (C₁₈-Mel/C₁₈-Cbl-Cy5BS) undergo ready 660 nm-triggered lysis (66±2% relative to Triton X-100-treated RBCs). This result suggests that fluorophores can be used to tune the hemolytic sensitivity of RBCs to specific wavelengths, potentially enabling the release of different proteins from different RBC carriers in a wavelength-dependent fashion.

Spatially resolved photorelease using RBCs internally loaded with BSA-Alexa Fluor 647 (FIG. 2B) and externally loaded with C₁₈-Mel and C₁₈-Cbl-BS was demonstrated. In this study two controls were also employed; RBCs externally loaded with C₁₈-Cbl-BS only as well as RBCs that were not externally loaded (“Blank”). The combination of C₁₈-Mel/C₁₈-Cbl-BS on the surface and BSA-Alexa Fluor 647 in the interior, allowed imaging of intact RBCs at 635 nm without triggering hemolysis. The RBC samples were exposed to 515 nm (confocal microscopy) in a spatially well-defined fashion (ROI in dashed box). As expected, RBCs lacking the C₁₈-Mel/C₁₈-Cbl-BS trigger retained BSA-Alexa Fluor 647 fluorescence in both the 515 nm illuminated and non-illuminated regions (FIG. 2B, top row). Similarly, RBCs surface labelled with C₁₈-Cbl-BS alone were likewise stable to exposure to 515 nm (FIG. 2B, middle row), demonstrating that melittin was required to observe hemolysis. Finally, 515 nm-exposed RBCs containing both C₁₈-Cbl-BS and C₁₈-Mel undergo rapid photo-hemolysis as evidenced by the loss of internal fluorescence at 635 nm (FIG. 2B, bottom row). It is important to note the release of BSA fluorescence is only observed in the region of cells exposed to 515 nm light, thereby demonstrating that spatially controlled photo-hemolysis is feasible. In addition, a subset of these RBCs were exposed at an 8-fold reduced laser power setting (FIG. 2C). As expected, photo-hemolysis is still observed but to a lesser extent than that detected at the higher laser setting. These results were consistent with the notion that light modulation can control both the locus and the extent of protein release.

### Example 3 Photoactive Composition

In addition to RBCs, melittin is also capable of binding to and lysing a variety of other lipid membranes. To further expand the potential utility of the Mel-based photo-trigger, the controlled photolysis of liposomes was investigated. ₁₈-Cbl-BS and C₁₈-Mel were introduced onto the surface of liposomes containing internally loaded 5(6)-carboxyfluorescein, which was fluorescently quenched at the 100 mM loading concentration. Several controls were also examined, including liposomes that were not surface modified (FIG. 2D, “Blank”) those containing only C₁₈-Cbl-BS, and liposomes exposed to C₁₈-Mel. Illumination of liposome/blank and liposome/C₁₈-Cbl-BS failed to elicit a lytic response. By contrast, exposure of liposomes to C₁₈-Mel generated a robust fluorescent increase, indicative of release of internally loaded 5(6)-carboxyfluorescein. Finally, liposomes containing both C₁₈-Mel and C₁₈-Cbl-BS were irradiated, and a response analogous to that of C₁₈-Mel-treated liposomes was observed, supporting the hypothesis that lipid membrane formulations are susceptible to photo-disruption by the Mel-based construct.

### Example 4—Photoactivated Release of Therapeutic Protein

The targeted delivery of Tissue Factor (TF) to tumors has been extensively studied for the treatment of cancer. TF promotes blood clotting as a key participant in a biochemical cascade that results in the activation of thrombin and the subsequent conversion of soluble fibrinogen to insoluble clot-forming fibrin. Consequently, TF and various derivatives have been explored as agents designed to starve tumors of their blood supply. However, the circulatory half-life is brief (<1 min). Thrombin could potentially be used in a fashion analogous to that of TF, but due to potentially devastating systemic side effects, thrombin is currently limited to use as a topical agent to prevent excessive bleeding during surgery. The delivery of such potent procoagulant proteins must be carefully controlled since clotting at unwanted sites can lead to deadly side effects such as myocardial infarction or stroke.

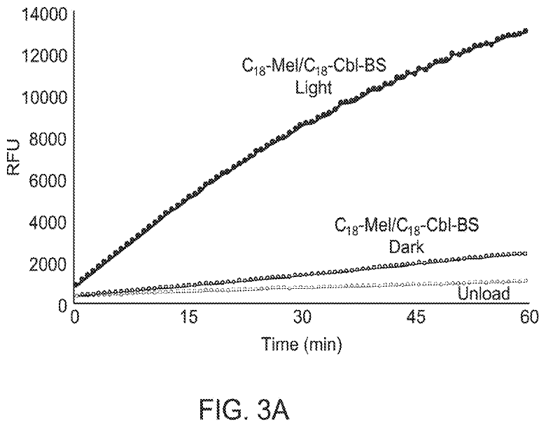

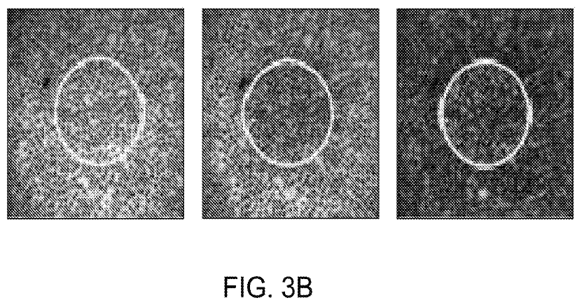

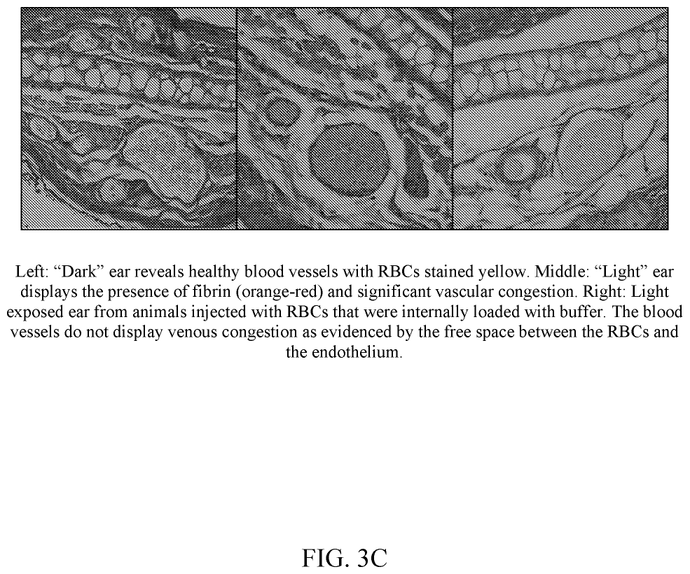

RBCs were prepared that were internally loaded with bovine thrombin and surface loaded with the photo-hemolytic C₁₈-Cbl-BS/C₁₈-Mel trigger. The proteolytic activity of photo-released thrombin was first examined using a fluorescent assay that employed a peptide-based thrombin substrate. Maximum thrombin release was established using C₁₈-Mel as a positive hemolytic control. Based on the latter, photo-hemolysis releases approximately 50% of total thrombin activity (FIG. 3A). By contrast, minimal thrombin activity was measured in the dark, which is comparable to the nearly negligible activity detected with RBCs that lack a photo-trigger.

The spatially directed thrombin photorelease was also examined using a fibrin polymerization assay. In brief, thrombin-catalyzed proteolysis of (Alexa Fluor 647)-fibrinogen generates fibrin, which polymerizes, resulting in an increase in Alexa Fluor 647 fluorescence. Both thrombin-loaded and -unloaded RBCs were suspended in solution with fluorescent fibrinogen and imaged using confocal microscopy (FIG. 3B). RBCs that were not loaded with thrombin were exposed to light (white circle) and, as expected, the fluorescence of the fibrinogen matrix remained unchanged over time. Illumination of thrombin loaded RBCs that contain only C₁₈-Cbl-BS on the cell surface, likewise, had no impact on fibrinogen matrix fluorescence. By contrast, the analogous experiment using thrombin-loaded RBCs containing the fully active photo-trigger, generated the anticipated fluorescence change that represents the conversion of fibrinogen to polymerized fibrin.

The spatially focused delivery of thrombin in vivo was subsequently examined. Healthy FVB mice were tail vein injected with either RBCs loaded with thrombin (experimental group) or only buffer (control group). In both cases, the RBCs were surface modified with a functional photo-hemolytic trigger. For both groups, a single ear was spot illuminated at 561 nm under a confocal microscope using an on-board laser. After light treatment, the animals were sacrificed and both light and dark exposed ears were collected. The ears were sectioned and subsequently stained with the Martius Scarlet Blue trichrome stain, which specifically labels collagen blue, RBCs yellow, and fibrin red (FIG. 3C). Mice injected with buffer-only RBCs displayed minimal fibrin staining in the blood vessels of both the light and dark ears. In addition, minimal fibrin staining is observed in blood vessels of the non-illuminated ears from mice injected with thrombin-loaded RBCs. By contrast, the vasculature in the light exposed ears exhibit a deep red stain indicative of profound fibrin formation.

### Experimental Details of the Examples

Materials: All materials were purchased from Sigma-Aldrich, Fisher Scientific, or VWR unless noted otherwise. Human red blood cells (hRBCs) were purchased from ZenBio. Mice were purchased from Jackson Laboratories.

Internal Loading of hRBCs

100 μL of hRBCs (less than 3 weeks old) were washed 3-5 times in fresh Leibovitz-15 media and then centrifuged at 1000×g for 3 min at room temperature. The supernatant of the final wash was removed, and the proper protein concentration was added so the final hematocrit was 70%. BSA-Fluorophore (BSA-FITC, BSA-Texas Red, or BSA-Alexa Fluor 647) had a final loading concentration of 4.0 mg/mL, bovine thrombin had a final loading of 5 NIH units, and TRAIL had a final loading concentration of 50 ug/mL. For mock loaded hRBCs, 43 μL of 1×PBS was added to the 100 μL hRBC pellet.

The protein-hRBC mixture was added to a prepared dialysis film with MWCO 1 kDa, clipped on both ends, and submerged in a 4° C. solution of 80 mOsm/L PBS+6 mM glucose while gently stirring for 20 min. The dialysis bag was then transferred to a 1×PBS solution at 37° C. for 10 min. The dialysis bag was washed with 900 μL of L-15 to transfer loaded RBCs to a new vial. The RBCs were washed 4-6 times in fresh L-15 at 1000×g for 3 min at room temperature.

hRBC Mel/BS External Loading

Protein loaded or mock loaded hRBCs (see Internal loading of hRBCs) were diluted to a concentration of 4.0×10⁸ cells/mL in L-15 media. Enough C₁₈-Melittin (C₁₈-Mel) was added to a fresh tube so the final concentration, after the addition of hRBCs, was 20 μM with a 1:2 ratio of C₁₈-Mel:C₁₈-Cbl-BS or a 1:4 ratio of C₁₈-Mel:C₁₈-Cbl-Cy5BS and left in the dark to equilibrate for 10 min. The corresponding amount of hRBCs was added to the C₁₈-Mel/C₁₈-Cbl-BS or C₁₈-Mel/C₁₈-Cbl-Cy5BS solution and incubated for 30 min in the dark. The photosensitive hRBCs were then washed 1-2 times by centrifuging 1000×g for 3 min and replacing supernatant with fresh L-15.

Mouse Red Blood Cell (mRBC) Collection and Isolation

Whole blood was collected from FVB mice via cardiac puncture on the same day the mRBCs were scheduled to be used. The whole blood was diluted 1:3 with 2 mM EDTA in 1×DPBS. 4 mL of whole blood was carefully pipetted to the top layer of 3 mL of sterile Ficoll-Paque Premium. The blood-Ficoll mixture was centrifuged for 30 min at 400×g. The mRBCs were then collected and washed with 1×PBS 3-5 times by centrifuging at 600×g for 2 min at 4° C.

Internal Loading of mRBCs

200-600 μL of mRBCs (see mRBC Collection and Isolation) were prepared in a single dialysis bag (MWCO 1 kDa). The proper protein concentration was added so the final hematocrit was 70%. BSA-Fluorophore (BSA-FITC or BSA-Texas Red) had a final loading concentration of 4.0 mg/mL and bovine thrombin had a final loading of 5 NIH units. For mock loaded mRBCs, 1×PBS was added to the mRBCs so the final hematocrit was 70%.

The protein/mRBC mixture was added to a prepared dialysis film with MWCO 1 kDa, clipped on both ends, and submerged in a 4° C. solution of 80 mOsm/L PBS, 10 mM glucose, 0.25% glycerol, and 2 mM ATP (added the day of loading) while stirring gently for 40 min. Cells were recovered from the dialysis bags with a small amount of 1×PBS (less than 400 μL) and added to a fresh tube. The solutions were brought to isotonic conditions with 10×PBS and then incubated at 37° C. for 20 min. The mRBCs were washed 4-6 times in fresh 1×PBS at 600×g for 2 min at 4° C.

mRBC MelBS External Loading

100 μL of protein loaded or mock loaded mRBCs (see Internal loading of mRBCs) were diluted to a 10% hematocrit in 1×PBS. Enough C₁₈-Mel was added to a fresh tube so the final concentration after the addition of mRBCs was 20 μM with a 1:2 ratio of C₁₈-Mel:C₁₈-Cbl-BS and left in the dark to equilibrate for 10 min. The mRBCs were added to the C₁₈-Mel/C₁₈-Cbl-BS solution and incubated for 30 min in the dark. The photosensitive mRBCs were then centrifuged at 600×g for 2 min and the supernatant was removed.

### Photosensitive RBC Lysis Assay

100 μL of 4.0×10⁸ photosensitive RBCs/mL internally loaded with BSA-Texas Red (see hRBC MelBS External Loading) was added to individual tubes containing 125 μL of L-15 or 125 μL of 1% Triton X-100 (positive control). Tubes were then incubated in the dark or under an LED board at 525 nm or 660 nm for 30 min. Cells were spun down for 3 min at 1000×g and supernatants were collected. The fluorescence was measured at Ex/Em 596/615 nm to determine release of BSA-Texas Red. BSA-Texas Red-hRBCs that were not externally loaded with C₁₈-Mel/C₁₈-Cbl-BS were illuminated for 30 min and used as a negative control. The fluorescence of L-15 alone was used as a blank control. Percent lysis was determined by the following equation: (Fluorescenceₛₐₘₚₗₑ−Fluorescence_{Blank})/(Fluorescenceₚₒₛᵢₜᵢᵥₑ−Fluorescence_{Blank})

### Thrombin Activity Assay

100 μL of 4.0×10⁸ photosensitive RBCs/mL internally loaded with thrombin (see hRBC MelBS External Loading) was added to individual tubes containing 125 μL of L-15 or 125 μL of 20 μM Mel (positive control). Tubes were then incubated in the dark or under an LED board at 525 nm for 30 min. Cells were spun down for 3 min at 1000×g and supernatants were collected. 20 μL of each supernatant were added to reaction mixtures with spiked Thrombin ranging from 0-0.5 Units/mL, 10 μM thrombin Fluorogenic Substrate III (Millipore), and 100 mM Tris pH 8.0 buffer. The fluorescence at an Ex/Em of 370/450 nm of these samples were measured every 45 s for 2 h. Standard additions curves were constructed to determine the activity of thrombin in each sample.

### Liposome Kinetic Assay

Custom DOPC:DOPE liposomes were purchased from Encapsula Nanosciences containing 100 mM 5(6)-carboxyfluorescein and a total lipid concentration of 10 mM. Prior to each experiment, excess 5(6)-carboxyfluorescein was removed from the liposomes using illustra Microspin G-50 columns. The columns were prepared according to the manufacturer's instructors, and 10 μL of liposomes were sent through the column. The cleaned liposomes were diluted 100× with L-15. Samples were prepared with 10 μM C₁₈-Mel, 30 μM C₁₈-Cbl-BS, 10 μM C₁₈-Mel and 30 μM C₁₈-Cbl-BS (adding the liposomes last), or just L-15. Fluorescence kinetics were acquired in a spectrofluorimeter using 494 nm, which is absorbed by both the Cbl (to effect lysis) and both the freed unquenched 5(6)-carboxyfluorescein (λₑₘ=515 nm). Data was collected every second for 8 min.

### MelBS Loading Concentration Assay

Mock loaded hRBCs were externally loaded with C₁₈-Cbl-Cy5BS as described in hRBC Mel/BS External Loading. The hRBCs (n=3) were 10× diluted to a final concentration of 0.1% Triton and exposed to 525 nm light for 1 h to ensure photolysis of the Cy5-BS peptide. The absorbance at 646 nm of the supernatant of these samples was measured to determine the amount of C₁₈-Cbl-Cy5-BS that was externally loaded to hRBCs. Mock loaded hRBCs (not externally loaded with C₁₈-Cbl-Cy5-BS) were used as a blank, and pure C₁₈-Cbl-Cy5-BS was used to determine the extinction coefficient of the photolyzed peptide.

### Confocal Microscopy

All imaging was performed on an inverted Olympus FV1000 scanning confocal microscope with an IX81 base. DiO, BSA-TexasRed and BSA-Alexa Fluor 647 were imaged with 488, 559 and 635 nm laser lines, respectively. Photolysis was performed using a 10 mW 515 nm laser line at 10-80% maximal power for indicated durations of photolysis at 10-100 μs/pixel dwell time.

### Imaging DiO TxRed-BSA Loaded RBCs

hRBCs were internally loaded with BSA-Texas Red as described in Internal loading of hRBCs. A subset of BSA-Texas Red hRBCs in 0.2% FBS L-15 at a 10% hematocrit were then externally loaded with DiO by incubating with 25 μM of DiO at 37° C. for 45 min. DiO/BSA-Texas Red loaded RBCs were imaged on the confocal microscope described in Confocal Microscopy with a 100× oil objective. Imaging was performed with 1024×1024 pixel resolution and 1.3× zoom with a 10 μs/pixel dwell time and 4× Kalman averaging by line. The DiO and TexasRed channels were excited by 488 and 559 laser lines respectively and imaged sequentially to minimize spectral bleed-through.

Photolytic Release of BSA-Alexa Fluor 647 from Loaded RBCs

RBCs loaded with BSA-Alexa Fluor 647 and C₁₈-Mel/C₁₈-Cbl-BS (see hRBCMelBS External Loading) were pipetted into channels of ibidi μ-slide VI 0.5 glass bottom slides coated with poly-1-lysine (Sigma) and equilibrated in the dark on the microscope for 5-10 min before imaging. The final RBC hematocrit was 1%. Images were acquired every 10 s. A single image was acquired before photolysis commenced followed by 6 more frames after photolysis. The photolysis ROI was defined to be the middle third of the field of view. Image analysis was performed in imageJ. Relative fluorescent intensity was calculated by dividing the mean fluorescent intensity of an ROI at time x by the mean fluorescent intensity at time 0.

### Fibrin Gel Assay

Alexa Fluor 647 conjugated fibrinogen (ThermoFisher) was reconstituted to 3 mg/mL in 0.1 M NaHCO₃ according to manufacturer's instructions. RBC free fibrin assays were performed at an Alexafluor 647-fibrinogen concentration of 1 mg/mL in fibrinogen assay buffer composed of 50 mM Tris pH 7.5, 0.1 M NaCl, and 20 mM CaCl₂). The fluorescent fibrinogen solution was initially plated on a glass bottom 35 mm dish (Mattek #1.5 glass coverslip) and imaged on the confocal microscope described in ConfocalMicroscopy. Thrombin was added to a final concentration of 0.05 U/μL and imaged 1 min later to assess the formation of fluorescent fibrin gel.

Photolytic Release of Thrombin from Loaded RBCs and Subsequent Fibrin Gel Formation

RBCs were internally loaded with thrombin and externally loaded with C₁₈-Mel/C₁₈-Cbl-BS as described in hRBC Mel/BS External Loading. A 1% hematocrit suspension of loaded RBCs was made in L-15 media containing 1 mg/mL Alexa Fluor 647 conjugated fibrinogen (ThermoFisher). The suspension was loaded into ibidi μ-slides as described in Photolytic release of BSA-Alexa Fluor 647 from Loaded RBCs. The suspensions were allowed to equilibrate in the dark for 5-10 min before imaging on the confocal microscope described in Confocal Microscopy. Images were acquired in a 512×512 pixel window with a 60× oil immersion objective and a dwell time of 10 μs/pixel. Z stacks were acquired at 1.34 μm intervals for a total of ten optical sections spanning 13.4 μm. Stacks were acquired in a time course where one full set of stacks was acquired, photolysis performed, and nine more sets of stacks followed. Photolysis was performed in a 250×250 pixel circular ROI at 80% 515 nm laser power for a total of 6 s.

In vivo Thrombin Release

Healthy FVB mice were housed in an approved Division of Comparative Medicine facility until time of injection. Mice were anesthetized with 2% isoflurane and placed on a heated stage (37° C.) to maintain their core body temperature throughout the experiment. Hair was removed from both ears using hair removal cream and the right ear was immobilized by two-sided tape on an aluminum block. Blood vessels were located using the green fluorescence channel on an Olympus IV-100 laser scanning confocal microscope, which was also utilized as a light source for mRBC activation. Prior to injection, mRBCs were mock loaded, BSA-Texas Red loaded, or thrombin loaded as described in InternalLoading of mRBCs. Mock loaded and thrombin loaded mRBCs were then externally loaded with C₁₈-Mel and C₁₈-Cbl-BS as described in mRBC Mel/BS External Loading. Mice were injected with 30 μL of mRBCs-BSA-Texas Red, 13 μL of 1×PBS, and 100 uL of either mRBCs-thrombin-Mel/BS (thrombin group, n=4) or mRBCs-Mock-Mel/BS (Mock group, n=4). The right ear of each mouse was then illuminated with a 488 nm laser at 30% intensity and a 561 nm laser at 45% intensity for 10 min to photoactive the mRBCs. After 90 min, mice were euthanized with CO₂ followed by a secondary physical method and both ears were harvested. All animal experimentation performed was approved by the Institutional Animal Care and Use Committee at the University of North Carolina at Chapel Hill.

### Histology Staining and Imaging

After collection of mouse ears (see In vivo Thrombin Release), the illuminated blood vessels in the right ears and the corresponding vessel in the non-illuminated ear were marked with a tissue staining dye. All ears were immediately placed in a 10% neutral buffered formalin solution for at least 48 h at room temperature. After fixation, the tissues were placed in a 70% ethanol solution and embedded in paraffin. 4 m cross-sections were stained with H&E and Martius Scarlet Blue dyes. Images were acquired using an Olympus BX51 microscope with a 40× objective.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A photoactive composition comprising:
a membrane-based carrier; and
a photoactivated lytic component coupled to the membrane-based carrier, the photoactivated lytic component comprising a lysing agent and photolabile lytic blocking agent.

2. The photoactive composition of claim 1, wherein the lysing agent and photolabile lytic blocking agent are coupled to the membrane of the carrier.

3. The photoactive composition of claim 1, wherein the photolabile lytic blocking agent comprises a photolytic moiety.

4. The photoactive composition of claim 3, wherein the photolytic moiety blocks activity of the lysing agent.

5. The photoactive composition of claim 3, wherein the photolytic moiety is a transition metal complex.

6. The photoactive composition of claim 1, wherein the membrane-based carrier comprises a lipid bilayer.

7. The photoactive composition of claim 1 further comprising one or more therapeutic agents in the membrane-based carrier.

8. The photoactive composition of claim 7, wherein the one or more therapeutic agents comprise biomolecules or pharmaceutical compositions.

9. The photoactive composition of claim 8, wherein the biomolecules comprise proteins, polysaccharides, peptides or nucleic acids.

10. The photoactive composition of claim 1, wherein the lysing agent and photolabile lytic blocking agent are independently coupled to the membrane-based carrier.

11. The photoactive composition of claim 1, wherein the lysing agent and photolabile lytic blocking agent are coupled to one another.

12. The photoactive composition of claim 1, wherein the photolabile lytic blocking agent absorbs light in the visible and/or infrared regions of the electromagnetic spectrum.

13. A therapeutic composition comprising:
a first photoactive composition including a membrane-based carrier containing a first therapeutic agent, and a photoactivated lytic component coupled to the membrane-based carrier, the photoactivated lytic component comprising a lysing agent and photolabile lytic blocking agent; and
a second photoactive composition including a membrane-based carrier containing a second therapeutic agent, and a photoactivated lytic component coupled to the membrane-based carrier, the photoactivated lytic component comprising a lysing agent and photolabile lytic blocking agent, wherein the photolabile blocking agents of the first and second photoactive compositions have differing absorption spectra.

14. The therapeutic composition of claim 13, wherein the first therapeutic agent and the second therapeutic agent are different.

15. The therapeutic composition of claim 13, wherein the first and second therapeutic agents work in conjunction to treat a diseased condition in a patient.

16. A method of treatment comprising:
delivering a photoactive composition to a diseased site, the photoactive composition comprising a membrane-based carrier containing one or more therapeutic agents, and a photoactivated lytic component coupled to the membrane-based carrier, the photoactivated lytic component comprising a lysing agent and photolabile lytic blocking agent;
activating the lysing agent via irradiating the photolabile lytic blocking agent; and
lysing the membrane-based carrier to release the one or more therapeutic agents at the diseased site.

17. The method of claim 16, wherein the lysing agent and photolabile lytic blocking agent are coupled to the membrane-based carrier membrane.

18. The method of claim 16, wherein the one or more therapeutic agents comprise biomolecules or pharmaceutical compositions.

19. The method of claim 18, wherein the biomolecules comprise proteins, polysaccharides, peptides, or nucleic acids.

20. The method of claim 18, wherein the pharmaceutical compositions comprise small molecule drugs.

21. The method of claim 18, wherein the membrane based carrier is selected from the group consisting of a cell, liposome, and exosome.

22. The method of claim 21, wherein the membrane-based carrier is an erythrocyte.

23. The method of claim 22, wherein the diseased site comprises a thrombus.

24. The method of claim 16 further comprising delivering an additional photoactive composition to the diseased site, the additional photoactive composition comprising a membrane-based carrier containing one or more additional therapeutic agents, and a photoactivated lytic component coupled to the membrane-based carrier, the photoactivated lytic component comprising a lysing agent and photolabile lytic blocking agent;
activating the lysing agent of the additional photoactive composition via irradiation of the photolabile lytic blocking agent; and
lysing the membrane-based carrier to release the one or more additional therapeutic agents at the diseased site.

25. The method of claim 24, wherein irradiation of the photolabile lytic blocking agents of the photoactive composition and the additional photoactive composition is administered sequentially.

26. The method of claim 24, wherein irradiation of the photolabile lytic blocking agents of the photoactive composition and the additional photoactive composition is administered simultaneously.

27. The method of claim 24, wherein the photolabile lytic blocking agents of photoactive composition and additional photoactive composition have different absorption spectra.

28. The method of claim 24, wherein the one or more therapeutic agents of the photoactive composition and the additional photoactive composition work in conjunction to treat the diseased site.

29. The method of claim 24, wherein the membrane-based carriers of the photoactive composition and the additional photoactive composition are erythrocytes.
